(19) United States
(12) Patent Application Publication
(10) Pub. No.: US 2013/0053717 A1
(43) Pub. Date: Feb. 28, 2013

(54) AUTOMATIC VENTILATOR CHALLENGE TO INDUCE SPONTANEOUS BREATHING EFFORTS

(71) Applicants: J. Doug Vandine, Manteca, CA (US); Peter R. Doyle, Vista, CA (US)

(72) Inventors: J. Doug Vandine, Manteca, CA (US); Peter R. Doyle, Vista, CA (US)

(73) Assignee: Nellcor Puritan Bennett LLC, Boulder, CO (US)

(21) Appl. No.: 13/220,850

(22) Filed: Aug. 30, 2011

(51) Int. Cl. A61B 5/097

(52) U.S. Cl. 600/532

(57) ABSTRACT

This disclosure describes systems and methods for configuring a ventilator for providing automatic, periodic ventilator adjustments to stimulate non-triggering patients to begin initiating spontaneous breathing efforts. Based on studies of respiration, a neural signal stimulating spontaneous breathing efforts is initiated when a patient's arterial partial pressure of carbon dioxide (PaCO₂) reaches a certain threshold level, which threshold level may vary from individual to individual. Accordingly, an automatic, periodic ventilator challenge to stimulate spontaneous breathing efforts in a non-triggering patient is provided. An automatic, periodic ventilator challenge refers to an automatic, periodic reduction in the ventilation of a non-triggering patient intended to increase PaCO₂ levels within a clinically-acceptable range. By periodically increasing PaCO₂ levels, the systems and methods disclosed herein seek to achieve the patient's individual PaCO₂ threshold level and, consequently, to evoke a neural signal stimulating spontaneous breathing efforts.

A ventilator is a device that mechanically helps patients breathe by replacing some or all of the muscular effort required to inflate and deflate the lungs. In recent years, there has been an accelerated trend towards an integrated clinical environment. That is, medical devices are becoming increasingly integrated with communication, computing, and control technologies. As a result, modern ventilatory equipment has become increasingly complex and highly efficient. In fact, modern ventilation has become so advanced and some patients may be so adequately ventilated that they fail to initiate spontaneous breathing efforts. Studies have shown that a patient's ability to initiate breaths is one of the key factors dictating the amount of time that the patient will remain on the ventilator. Moreover, mortality rates are shown to increase the longer patients remain on the ventilator.

Indeed, clinicians and patients may greatly benefit from automatic, periodic ventilator adjustments configured to stimulate non-triggering patients to begin initiating spontaneous breathing efforts.

### Automatic Ventilator Challenge to Induce Spontaneous Breathing Efforts

This disclosure describes systems and methods for configuring a ventilator for providing automatic, periodic ventilator adjustments to stimulate non-triggering patients to begin initiating spontaneous breathing efforts. Based on studies of respiration, a neural signal stimulating spontaneous breathing efforts is initiated when a patient's arterial partial pressure of carbon dioxide (PaCO₂) reaches a certain threshold level, which threshold level may vary from individual to individual. Due to advances in the ventilation of patients, some patients may be so adequately ventilated (i.e., the delivery of oxygen and the release of carbon dioxide is so efficient) that the level of carbon dioxide in the patient's blood (PaCO₂) does not reach the threshold level. As a result, the neural signal inducing spontaneous breathing efforts may not occur for those patients.

Accordingly, an automatic, periodic ventilator challenge to stimulate spontaneous breathing efforts in a non-triggering patient is provided. An automatic, periodic ventilator challenge refers to an automatic, periodic reduction in the ventilation of a non-triggering patient intended to increase PaCO₂ levels within a clinically-acceptable range. By periodically increasing PaCO₂ levels, the systems and methods disclosed herein seek to achieve the patient's individual PaCO₂ threshold level (also referred to as a patient-specific PaCO₂ threshold level) and, consequently, to evoke a neural signal stimulating spontaneous breathing efforts.

According to embodiments, a ventilator-implemented method for stimulating spontaneous breathing efforts when carbon dioxide monitoring is available is provided. The method comprises collecting ventilatory data associated with PaCO₂ or a surrogate for PaCO₂ and processing the ventilatory data to determine a first target PaCO₂ level. The method further comprises determining a first reduction in ventilation projected to achieve the first target PaCO₂ level and providing a first ventilator challenge based on the first reduction in ventilation. The method also comprises determining whether spontaneous breathing efforts are detected in response to the first ventilator challenge and, when spontaneous breathing efforts are detected, identifying a successful PaCO₂ level associated with the first ventilator challenge.

According to additional embodiments, a ventilator-implemented method for stimulating spontaneous breathing efforts when carbon dioxide monitoring is not available is provided. The method comprises determining a first test percentage for reducing ventilation and providing a first ventilator challenge based on the first test percentage for reducing ventilation. The method also comprises determining whether spontaneous breathing efforts are detected in response to the first ventilator challenge and, when spontaneous breathing efforts are detected, identifying a successful test percentage associated with the first ventilator challenge.

According to additional embodiments, a ventilator processing interface for stimulating spontaneous breathing efforts is provided. The ventilator processing interface comprising means for determining a first test percentage for reducing ventilation to stimulate spontaneous breathing efforts and means for providing a first ventilator challenge based on the first test percentage for reducing ventilation. The ventilator processing interface comprising means for determining whether spontaneous breathing efforts are detected in response to the first ventilator challenge and, when spontaneous breathing efforts are detected, means for identifying a successful test percentage associated with the first ventilator challenge.

These and various other features as well as advantages which characterize the systems and methods described herein will be apparent from a reading of the following detailed description and a review of the associated drawings. Additional features are set forth in the description which follows, and in part will be apparent from the description, or may be learned by practice of the technology. The benefits and features of the technology will be realized and attained by the structure particularly pointed out in the written description and claims hereof as well as the appended drawings.

It is to be understood that both the foregoing general description and the following detailed description are exemplary and explanatory and are intended to provide further explanation of the claims.

### BRIEF DESCRIPTION OF THE DRAWINGS

The following drawing figures, which form a part of this application, are illustrative of described technology and are not meant to limit the scope of the claims in any manner, which scope shall be based on the claims appended hereto.

FIG. 1 is a diagram illustrating an embodiment of an exemplary ventilator connected to a human patient.

FIG. 2 is a block-diagram illustrating an embodiment of a ventilatory system for providing an automatic, periodic ventilator challenge to stimulate spontaneous breathing efforts.

FIG. 3 is a flow chart illustrating an embodiment of a method for providing a first ventilator challenge to stimulate spontaneous breathing efforts when carbon dioxide monitoring is available.

FIG. 4 is a flow chart illustrating an embodiment of a method for providing a second ventilator challenge to stimulate spontaneous breathing efforts when carbon dioxide monitoring is available.

FIG. 5 is a flow chart illustrating an embodiment of a method for providing a first ventilator challenge to stimulate spontaneous breathing efforts when carbon dioxide monitoring is not available.

FIG. 6 is a flow chart illustrating an embodiment of a method for providing a second ventilator challenge to stimulate spontaneous breathing efforts when carbon dioxide monitoring is not available.

### DETAILED DESCRIPTION

Although the techniques introduced above and discussed in detail below may be implemented for a variety of medical devices, the present disclosure will discuss the implementation of these techniques for use in a mechanical ventilator system. The reader will understand that the technology described in the context of a ventilator system could be adapted for use with other therapeutic equipment for making minor adjustments to patient care to induce a patient response.

This disclosure describes systems and methods for configuring a ventilator for providing automatic, periodic ventilator adjustments to stimulate non-triggering patients to begin initiating spontaneous breathing efforts. Based on studies of respiration, a neural signal stimulating spontaneous breathing efforts is initiated when a patient's arterial partial pressure of carbon dioxide (PaCO₂) reaches a certain threshold level. The threshold level of PaCO₂ may vary from individual to individual, but whenever the threshold level is reached a neural signal inducing spontaneous breathing efforts occurs. Due to advances in the ventilation of patients, some patients may be so adequately ventilated (i.e., the delivery of oxygen and the release of carbon dioxide is so efficient) that the level of carbon dioxide in the patients' blood (PaCO₂) does not reach the threshold level. As a result, the neural signal inducing spontaneous breathing efforts may not occur for those patients.

According to embodiments, a ventilator may be configured to provide an automatic, periodic ventilator challenge to stimulate spontaneous breathing efforts in a non-triggering patient. An automatic, periodic ventilator challenge refers to an automatic, periodic reduction in the ventilation of a non-triggering patient intended to increase PaCO₂ levels within a clinically-acceptable range. By periodically increasing PaCO₂ levels, the systems and methods disclosed herein seek to achieve the patient's individual PaCO₂ threshold level and, consequently, to evoke a neural signal stimulating spontaneous breathing efforts.

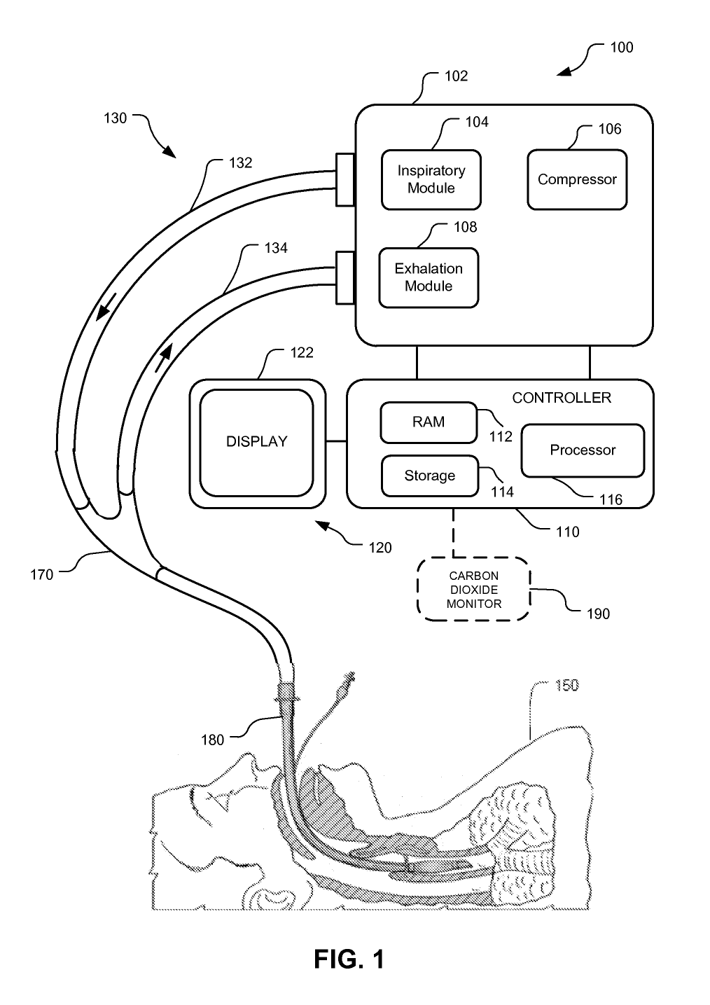

FIG. 1 is a diagram illustrating an embodiment of an exemplary ventilator 100 connected to a human patient 150.

Ventilator 100 includes a pneumatic system 102 (also referred to as a pressure generating system 102) for circulating breathing gases to and from patient 150 via the ventilation tubing system 130, which couples the patient to the pneumatic system via an invasive (e.g., endotracheal tube, as shown) or a non-invasive (e.g., nasal mask) patient interface.

Ventilation tubing system 130 may be a two-limb (shown) or a one-limb circuit for carrying gases to and from the patient 150. In a two-limb embodiment, a fitting, typically referred to as a “wye-fitting” 170, may be provided to couple a patient interface 180 (as shown, an endotracheal tube) to an inspiratory limb 132 and an expiratory limb 134 of the ventilation tubing system 130.

Pneumatic system 102 may be configured in a variety of ways. In the present example, system 102 includes an exhalation module 108 coupled with the expiratory limb 134 and an inhalation module 104 coupled with the inspiratory limb 132. Compressor 106 or other source(s) of pressurized gases (e.g., air, oxygen, and/or helium) is coupled with inhalation module 104 to provide a gas source for ventilatory support via inspiratory limb 132.

The pneumatic system 102 may include a variety of other components, including mixing modules, valves, sensors, tubing, accumulators, filters, etc. Controller 110 is operatively coupled with pneumatic system 102, signal measurement and acquisition systems, and an operator interface 120 that may enable an operator to interact with the ventilator 100 (e.g., change ventilatory settings, select operational modes, view monitored parameters, etc.). Controller 110 may include memory 112, one or more processors 116, storage 114, and/or other components of the type commonly found in command and control computing devices. In the depicted example, operator interface 120 includes a display 122 that may be touch-sensitive and/or voice-activated, enabling the display 122 to serve both as an input and output device.

In addition, according to some embodiments, pneumatic system 102 may optionally be communicatively coupled to a carbon dioxide monitor 190. According to some embodiments, carbon dioxide monitor 190 may be an arterial blood gas (ABG) monitor. An ABG monitor may be any suitable device for detecting an arterial partial pressure of carbon dioxide (PaCO₂). PaCO₂ is a measure of the various forms of carbon dioxide (e.g., bicarbonate and carbamino compounds) dissolved in the blood. For a normal individual, PaCO₂ ranges between about 36 and 44 mmHg. For example, an ABG monitor may periodically or continuously access and analyze arterial blood from patient 150. Arterial blood may be accessed by the ABG monitor via any suitable means, including but not limited to drawing arterial blood through au arterial catheter (attachment to patient 150 not shown) inserted into any suitable artery (e.g., the radial artery at the wrist, the femoral artery in the groin, dorsalis pedis artery in the foot, etc.).

According to alternative embodiments, carbon dioxide monitor 190 may be a capnography device that periodically or continuously monitors the exhaled gases of patient 150. Exhaled gases may be monitored by the capnography device via any suitable means, including but not limited to monitoring gases in the expiratory limb 134 (not shown) or at or near the expiratory valve (not shown). Alternatively or additionally, the capnography device may be an internal device (not shown) and may monitor exhaled gases by communicating with the exhalation module 108. By measuring the exhaled gases of a patient, the capnography device may determine the end tidal carbon dioxide (EtCO₂), which is a measure of the volume of carbon dioxide exhaled by the patient. EtCO₂ is a surrogate for PaCO₂ and an estimate of PaCO₂ may be derived from EtCO₂. As used herein, a surrogate for PaCO₂ is any measurement or estimate of carbon dioxide level that may be used to estimate or derive PaCO₂.

According to alternative embodiments, carbon dioxide monitor 190 may be a transcutaneous monitoring device. The transcutaneous monitoring device may periodically or continuously monitor transcutaneous carbon dioxide (PtcCO₂) levels. Transcutaneous monitoring devices are non-invasive and rely on a heating element and an electrode to detect the partial pressure of carbon dioxide in the tissues of the skin (attachment to patient 150 not shown). When corrected for normal body temperatures, PtcCO₂ approximates PaCO₂ but may measure slightly higher than PaCO₂ due to CO₂ production in the tissues. However a relationship between PtcCO₂ and PaCO₂ may be established for a particular patient and may be used to convert PtcCO₂ measurements to PaCO₂. This method may reduce or eliminate the need to invasively access the patient's arterial blood.

According to alternative embodiments, carbon dioxide monitor 190 may be a mixed venous blood monitoring device. The mixed venous blood monitoring device may be configured to monitor the mixed venous partial pressure of carbon dioxide (PvCO₂) via any suitable means. PvCO₂ is a measure of the partial pressure of carbon dioxide in the pulmonary capillaries prior to gas exchange with the lungs. As such, the PvCO₂ level for a normal individual is about 6 mm Hg greater than a measure of PaCO₂ for that individual. PvCO₂ may be monitored periodically or continuously by accessing and analyzing mixed venous blood from patient 150 (attachment to patient 150 not shown).

According to alternative embodiments, carbon dioxide monitor 190 may be an alveolar monitoring device. The alveolar monitoring device may be configured to monitor the alveolar partial pressure of carbon dioxide (P_{A}CO₂). P_{A}CO₂ is a measure of the partial pressure of carbon dioxide in the alveoli of the lungs and is statistically equivalent to PaCO₂. This is true because, based on a pressure gradient between PvCO₂ and P_{A}CO₂, carbon dioxide passes from the mixed venous blood of the pulmonary capillaries and into the lungs, where it is exhaled. Therefore, upon pressure equilibrium with the alveoli, arterial blood leaving the lungs exhibits a lower partial pressure of carbon dioxide than mixed venous blood entering the pulmonary capillaries and exhibits roughly the same partial pressure of carbon dioxide as the alveoli. P_{A}CO₂ may be monitored periodically or continuously by accessing and analyzing the alveoli of patient 150 (attachment to patient 150 not shown).

According to still alternative embodiments, pneumatic system 102 may not be configured to periodically or continuously monitor carbon dioxide levels either directly or indirectly.

The memory 112 includes non-transitory, computer-readable storage media that stores software that is executed by the one or more processors 116 and which controls the operation of the ventilator 100. In an embodiment, the memory 112 includes one or more solid-state storage devices such as flash memory chips. In an alternative embodiment, the memory 112 may be mass storage connected to the one or more processors 116 through a mass storage controller (not shown) and a communications bus (not shown). Although the description of computer-readable media contained herein refers to a solid-state storage, it should be appreciated by those skilled in the art that computer-readable storage media can be any available media that can be accessed by the one or more processors 116. That is, computer-readable storage media includes non-transitory, volatile and non-volatile, removable and non-removable media implemented in any method or technology for storage of information such as computer-readable instructions, data structures, program modules or other data. For example, computer-readable storage media includes RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

Communication between components of the ventilatory system or between the ventilatory system and other therapeutic equipment and/or remote monitoring systems may be conducted over a distributed network, as described further herein, via wired or wireless means. Further, the present methods may be configured as a presentation layer built over the TCP/IP protocol. TCP/IP stands for “Transmission Control Protocol/Internet Protocol” and provides a basic communication language for many local networks (such as intra- or extranets) and is the primary communication language for the Internet. Specifically, TCP/IP is a hi-layer protocol that allows for the transmission of data over a network. The higher layer, or TCP layer, divides a message into smaller packets, which are reassembled by a receiving TCP layer into the original message. The lower layer, or IP layer, handles addressing and routing of packets so that they are properly received at a destination.

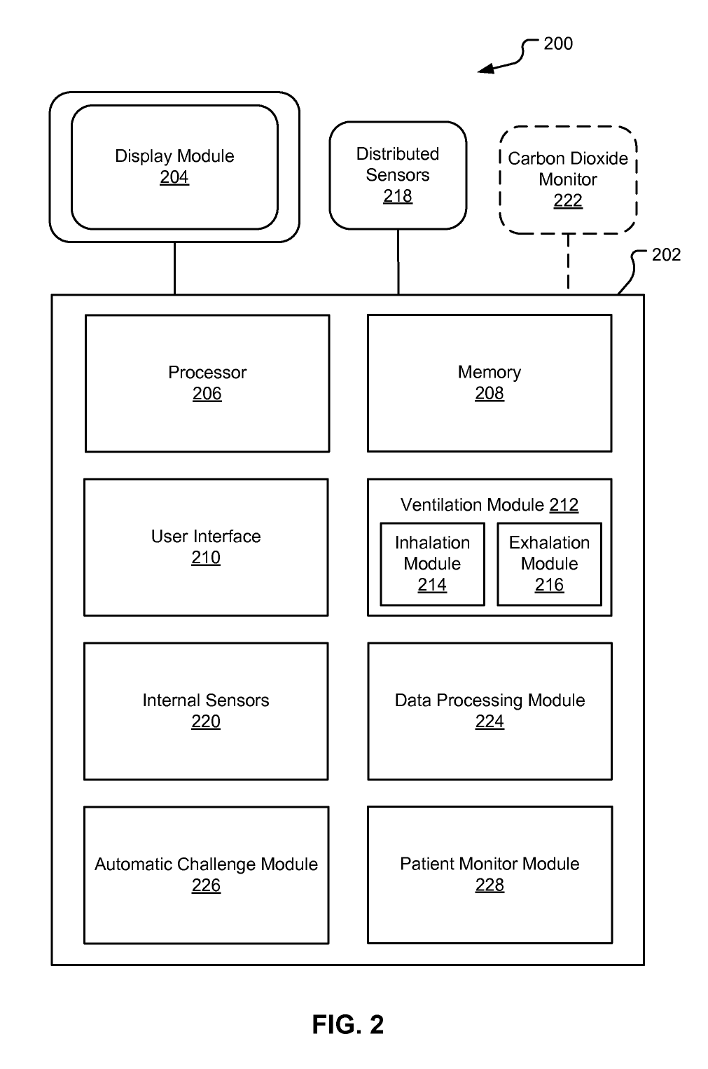

FIG. 2 is a block-diagram illustrating an embodiment of a ventilatory system for providing an automatic, periodic ventilator challenge to stimulate spontaneous breathing efforts.

Ventilatory system 200 includes ventilator 202 with its various modules and components. That is, ventilator 202 may further include, inter alia, one or more processors 206, memory 208, user interface 210, and ventilation module 212 (which may further include and/or communicate with inspiration module 214 and exhalation module 216). The one or more processors 206 are defined as described above for one or more processors 116. Processors 206 may further be configured with a clock whereby elapsed time may be monitored by the system 200. Memory 208 is defined as described above for memory 112.

The ventilatory system 200 may also include a display module 204 communicatively coupled to ventilator 202. Display module 204 may provide various input screens, for receiving clinician input, and various display screens, for presenting useful information to the clinician. The display module 204 is configured to communicate with user interface 210 and may include a graphical user interface (GUI). The GUI may be an interactive display, e.g., a touch-sensitive screen or otherwise, and may provide various windows (i.e., visual areas) comprising elements for receiving user input and interface command operations and for displaying ventilatory information (e.g., ventilatory data, alerts, patient information, parameter settings, etc.). The elements may include controls, graphics, charts, tool bars, input fields, etc. Alternatively, other suitable means of communication with the ventilator 202 may be provided, for instance by a wheel, keyboard, mouse, or other suitable interactive device. Thus, user interface 210 may accept commands and input through display module 204. Display module 204 may also provide useful information in the form of various ventilatory data regarding the physical condition of a patient and/or a prescribed respiratory treatment. The useful information may be derived by the ventilator 202, based on data collected by a data processing module 224, and the useful information may be displayed to the clinician on display module 204 in the form of graphs, wave representations, pie graphs, or other suitable forms of graphic display.

Ventilation module 212 may oversee ventilation of a patient according to ventilatory settings. Ventilatory settings may include any appropriate input for configuring the ventilator to deliver breathable gases to a particular patient. Ventilatory settings may be entered by a clinician, e.g., based on a prescribed treatment protocol for the particular patient, or automatically generated by the ventilator, e.g., based on attributes (i.e., age, diagnosis, ideal body weight, gender, etc.) of the particular patient according to any appropriate standard protocol or otherwise. For example, ventilatory settings may include, inter alia, tidal volume (V_{T}), minute ventilation (\dot{V}_{E}), respiratory rate (RR), inspiratory time (T_{I}), inspiratory pressure (P_{I}), pressure support (P_{SUPP}), rise time percent (rise time %), peak flow, flow pattern, etc.

As used herein, ventilation generally refers to the movement of gases into and out of the lungs. Minute ventilation (\dot{V}_{E}) refers to the volume of gases moved into or out of the lungs per minute and is generally represented in liter per minute (L/min or lpm). \dot{V}_{E} may be set by the clinician and is represented by the following equation:

\dot{V}_{E} (L/min)=RR (breaths/min)*V_{T} (L/breath)

In the above equation, RR is the respiratory rate (or frequency) and V_{T} is the tidal volume.

Ventilation module 212 may further include an inspiration module 214 configured to deliver gases to the patient according to prescribed ventilatory settings. Specifically, inspiration module 214 may correspond to the inhalation module 104 or may be otherwise coupled to source(s) of pressurized gases (e.g., air, oxygen, and/or helium), and may deliver gases to the patient. Inspiration module 214 may be configured to provide ventilation according to various ventilatory types and modes, e.g., via volume-targeted, pressure-targeted, or via any other suitable type of ventilation. Inspiration module 214 may be configured to deliver gases to the patient for a period of time, referred to as the inspiratory time (T_{I}). For a non-triggering patient, T_{I} may be set by the clinician or derived from the respiratory rate (RR) setting. For a triggering patient, T_{I} is based on the natural respiratory rate of the patient (as detected by inspiratory flow reaching a threshold percentage of peak flow, by patient expiratory efforts, or otherwise).

Ventilation module 212 may further include an exhalation module 216 configured to release gases from the patient's lungs according to prescribed ventilatory settings. Specifically, exhalation module 216 may correspond to exhalation module 108 or may otherwise be associated with and/or control an exhalation valve for releasing gases from the patient. By way of general overview, a ventilator may initiate exhalation based on lapse of an inspiratory time setting (T_{I}) or other cycling criteria set by the clinician or derived from ventilatory settings (e.g., detecting delivery of prescribed V_{T} or prescribed P_{I} based on a reference trajectory). Alternatively, exhalation may be cycled based on detection of patient effort or otherwise. Upon initiating the exhalation phase, exhalation module 216 may allow the patient to exhale by opening an exhalation valve. As such, exhalation is passive, and the direction of airflow, as described above, is governed by the pressure gradient between the patient's lungs (higher pressure) and the ambient surface pressure (lower pressure). Although expiratory flow is passive, it may be regulated by the ventilator based on the size of the exhalation valve opening. Indeed, the ventilator may regulate the exhalation valve in order to target set PEEP by applying a number of calculations and/or trajectories.

For a spontaneously-breathing patient, expiratory time (T_{E}) is the time from the end of inspiration until the patient triggers the next inspiration. For a non-spontaneously-breathing patient, it is the time from the end of inspiration until the next inspiration based on the set T_{I} and set RR. As may be further appreciated, at the point of transition between inspiration and exhalation, the direction of airflow may abruptly change from flowing into the lungs to flowing out of the lungs or vice versa depending on the transition.

According to some embodiments, the inspiration module 214 and/or the exhalation module 216 may be configured to synchronize ventilation with a spontaneously-breathing, or triggering, patient. That is, the ventilator may be configured to detect patient effort and may initiate a transition from exhalation to inhalation (or from inhalation to exhalation) in response. Triggering refers to the transition from exhalation to inhalation in order to distinguish it from the transition from inhalation to exhalation (referred to as cycling). Ventilation systems, depending on their mode of operation, may trigger and/or cycle automatically, or in response to a detection of patient effort, or both.

In some cases, the ventilatory settings described above may provide over-ventilation to a patient, i.e., the ventilator may provide such efficient exchange of carbon dioxide and oxygen that PaCO₂ levels never reach a threshold level. To address this issue, the systems and methods disclosed herein propose an automatic, periodic reduction in the ventilation of a non-triggering patient intended to increase PaCO₂ levels within a clinically-acceptable range. According to some embodiments, the minute ventilation (\dot{V}_{E}) of the patient may be reduced in order to cause a corresponding increase in PaCO₂. For instance, with reference to the equation above, \dot{V}_{E} may be reduced by decreasing RR and/or by decreasing V_{T}. It may be preferable to decrease \dot{V}_{E} by decreasing the RR; however, \dot{V}_{E} may also be decreased by additionally or alternatively decreasing the V_{T}. According to other embodiments, ventilation may be decreased by any other suitable means. According to some embodiments, the ventilation of the patient may be reduced for a period of time (e.g., 3 minutes, 5 minutes, 7 minutes) in order to cause a corresponding increase in PaCO₂. According to additional or alternative embodiments, the reduction in ventilation may be discontinued or cancelled before expiration of the period of time when the ventilator detects various other threshold conditions, for example, low oxygen saturation (SpO₂), increased heart rate, or any indication that the patient is responding adversely to the reduction in ventilation.

The ventilatory system 200 may also include one or more distributed sensors 218 communicatively coupled to ventilator 202. Distributed sensors 218 may communicate with various components of ventilator 202, e.g., ventilation module 212, internal sensors 220, data processing module 224, automatic challenge module 226, patient monitor module 228, and any other suitable components and/or modules. Distributed sensors 218 may be placed in any suitable location, e.g., within the ventilatory circuitry or other devices communicatively coupled to the ventilator. For example, sensors may be affixed to the ventilatory tubing or may be imbedded in the tubing itself. According to some embodiments, sensors may be provided at or near the lungs (or diaphragm) for detecting a pressure in the lungs. Additionally or alternatively, sensors may be affixed or imbedded in or near wye-fitting 170 and/or patient interface 180, as described above. Distributed sensors 218 may include pressure transducers for detecting circuit pressure, flowmeters for detecting circuit flow, optical or ultrasound sensors for measuring gas characteristics or other parameters, or any other suitable sensory device.

Ventilator 202 may further include one or more internal sensors 220. Similar to distributed sensors 218, internal sensors 220 may communicate with various components of ventilator 202, e.g., ventilation module 212, data processing module 224, automatic challenge module 226, patient monitor module 228, and any other suitable components and/or modules. Internal sensors 220 may employ any suitable sensory or derivative technique for monitoring one or more parameters associated with the ventilation of a patient. However, as opposed to the distributed sensors 218, the internal sensors 220 may be placed in any suitable internal location, such as, within the ventilatory circuitry or within components or modules of ventilator 202. For example, sensors may be coupled to the inhalation and/or exhalation modules for detecting pressure and/or flow. Specifically, internal sensors may include pressure transducers and flowmeters for measuring changes in pressure and airflow. Additionally or alternatively, internal sensors may utilize optical or ultrasound techniques for measuring changes in ventilatory parameters. For example, a patient's expired gases may be monitored by a capnography device to detect end tidal carbon dioxide (EtCO₂).

Ventilator 202 may further be optionally coupled to a carbon dioxide monitor 222. As described above with reference to carbon dioxide monitor 190, carbon dioxide monitor 222 may be an arterial blood gas (ABG) monitor for measuring PaCO₂, a capnography device for measuring EtCO₂, a transcutaneous monitoring device for measuring PtcCO₂, a mixed venous blood monitoring device for measuring PvCO₂, an alveolar monitoring device for measuring P_{A}CO₂, or any other suitable device for measuring PaCO₂ or a surrogate for PaCO₂. According to embodiments disclosed herein, EtCO₂, PtcCO₂, PvCO₂, and P_{A}CO₂ are all surrogates for PaCO₂, although other ventilatory parameters associated with carbon dioxide levels may also be considered surrogates for PaCO₂. According to alternative embodiments, Ventilator 202 is not coupled to a carbon dioxide monitor and is not configured to measure PaCO₂ or a surrogate for PaCO₂.

Ventilator 202 may further include a data processing module 224. As noted above, distributed sensors 218, internal sensors 220, and carbon dioxide monitor 222 may collect data regarding various ventilatory parameters. A ventilatory parameter refers to any factor, characteristic, or measurement associated with the ventilation of a patient, whether monitored by the ventilator or by any other device. For example, PaCO₂ and surrogates for PaCO₂ may be referred to as ventilatory parameters. Internal and distributed sensors and/or the carbon dioxide monitor may further transmit collected data to the data processing module 224 and, according to embodiments, the data processing module 224 may be configured to collect data regarding some ventilatory parameters, to derive data regarding other ventilatory parameters, and/or to graphically represent collected and/or derived data on display module 204. According to embodiments, any collected, derived, and/or graphically represented data may be referred to as ventilatory data. For example, collected, derived, and/or graphically represented data associated with PaCO₂ and surrogates for PaCO₂ may be referred to as ventilatory data.

For example, according to some embodiments, the ventilator may periodically or continuously collect ventilatory data associated with PaCO₂ (e.g., using an ABG monitor or other suitable device), EtCO₂ (e.g., using a capnography device or other suitable device), PtcCO₂ (e.g., using a transcutaneous monitoring device or other suitable device), PvCO₂ (e.g., using a mixed venous blood monitoring device), P_{A}CO₂ (e.g., using an alveolar monitoring device), or any other suitable measurement of carbon dioxide. According to some embodiments, the ventilator may periodically or continuously derive ventilatory data associated with PaCO₂. For instance, PaCO₂ may be indirectly determined or estimated based on measurements of EtCO₂, PtcCO₂, PvCO₂, P_{A}CO₂, or any other suitable measurement of carbon dioxide. According to some embodiments, ventilator 202 is not coupled to a carbon dioxide monitor and is not configured to collect and/or derive ventilatory data associated with PaCO₂ or surrogates for PaCO₂.

Ventilator 202 may further include an automatic challenge module 226. According to studies, when PaCO₂ levels reach a threshold level, a neural signal is initiated that stimulates spontaneous breathing efforts. The actual PaCO₂ threshold level may vary from patient to patient. As it relates to patient ventilation, in some cases, a ventilator may over-ventilate a patient when the exchange of oxygen and carbon dioxide is so efficient that PaCO₂ levels do not reach the threshold level for that patient. The term over-ventilate or over-ventilation means that the patient's respiratory needs are so well met by the ventilator, the patient is not stimulated to breath naturally. According to studies, when a patient does not breathe spontaneously for as few as 18 hours to 3 days, diaphragm myofibers may show significant atrophy. Furthermore, it takes significant time to regain diaphragm muscle strength following atrophy. As a result, a non-triggering patient may ultimately spend more time on the ventilator, which has been linked to higher mortality rates.

According to embodiments, the automatic challenge module 226 may provide an automatic, periodic ventilator challenge intended to stimulate a non-triggering patient to initiate spontaneous breathing efforts. According to embodiments, the ventilator challenge may temporarily reduce ventilation in order to cause a concomitant increase in arterial carbon dioxide (PaCO₂). That is, the ventilator challenge temporarily reduces ventilation from baseline ventilatory settings as configured for the patient. According to embodiments, if the increase in PaCO₂ reaches a patient-specific PaCO₂ threshold level, the non-triggering patient will be stimulated to initiate spontaneous breathing efforts.

According to some embodiments, the ventilator may be equipped to measure PaCO₂ or a surrogate for PaCO₂. In this case, ventilatory data associated with PaCO₂ or a surrogate for PaCO₂ may be collected or derived and a first ventilator challenge may be configured to reduce ventilation to achieve a first target PaCO₂ level. According to embodiments, PaCO₂ or a surrogate for PaCO₂ may be measured during or at the end of the first ventilator challenge. If spontaneous breathing efforts are initiated in response to the first ventilator challenge, the measurement of PaCO₂ or a surrogate for PaCO₂ may approximate the patient-specific PaCO₂ threshold level for that patient. According to embodiments, the first target PaCO₂ level (e.g., a successful PaCO₂ level) may be targeted in subsequent ventilator challenges until the patient consistently initiates spontaneous breathing efforts.

Alternatively, if spontaneous breathing efforts are not initiated in response to the first ventilator challenge, the measurement of PaCO₂ or a surrogate for PaCO₂ may approximate an unsuccessful PaCO₂ level below the patient-specific PaCO₂ threshold level. In this case, a second ventilator challenge may be configured to reduce ventilation to achieve a second target PaCO₂ level higher than the unsuccessful PaCO₂ level. According to embodiments, the target PaCO₂ level may be increased in subsequent ventilator challenges, within an acceptable range, until the patient initiates spontaneous breathing efforts. The acceptable range for target PaCO₂ levels may be based on the patient's disease state or other appropriate criteria.

According to embodiments, a successful PaCO₂ level may be targeted in subsequent ventilator challenges until the patient consistently initiates spontaneous breathing efforts. Alternatively or additionally, baseline ventilatory settings may be adjusted such that new ventilatory settings generally reduce ventilation to allow PaCO₂ levels to consistently reach the successful PaCO₂ level (an approximation of the patient-specific PaCO₂ threshold level) in order to prevent over-ventilation of the patient. For example, the new ventilatory settings may include a lower \dot{V}_{E} setting. Moreover, if the patient begins to consistently breathe spontaneously, the new ventilatory settings may include switching to a spontaneous mode of ventilation. New ventilatory settings may be input by the clinician or automatically adjusted by the ventilator. According to some embodiments, upon configuring new ventilatory settings, ventilator challenges may be discontinued unless and until the patient fails to initiate spontaneous breathing efforts.

According to alternative embodiments, the ventilator may not be equipped to measure PaCO₂ or a surrogate for PaCO₂. In this case, a first ventilator challenge may be configured to reduce ventilation by a first test percentage, e.g., about 10%. If spontaneous breathing efforts are initiated in response to the first ventilator challenge, the first test percentage reduction in ventilation (a successful test percentage reduction) may be maintained for subsequent ventilator challenges until the patient consistently initiates spontaneous breathing efforts. Alternatively, if spontaneous breathing efforts are not initiated in response to the first ventilator challenge, ventilation may be reduced by a second test percentage that is incrementally greater than the first test percentage, e.g. about 15%. According to embodiments, a percentage reduction in ventilation may be increased in subsequent ventilator challenges, within an acceptable range, until the patient initiates spontaneous breathing efforts. The acceptable range for the percentage reduction in ventilation may be based on the patient's disease state or other appropriate criteria.

According to embodiments, a successful test percentage may be targeted in subsequent ventilator challenges until the patient consistently initiates spontaneous breathing efforts. Alternatively or additionally, baseline ventilatory settings may be adjusted such that new ventilatory settings generally reduce ventilation by the successful test percentage in order to stimulate spontaneous breathing efforts and prevent over-ventilation of the patient. For example, the new ventilatory settings may include decreasing ventilation by the successful test percentage. Moreover, if the patient begins to consistently breathe spontaneously, the new ventilatory settings may include switching to a spontaneous mode of ventilation. New ventilatory settings may be input by the clinician or automatically adjusted by the ventilator. According to some embodiments, upon configuring new ventilatory settings, ventilator challenges may be discontinued unless and until the patient fails to initiate spontaneous breathing efforts.

For example, according to embodiments, the automatic challenge module 226 may provide an automatic, periodic ventilator challenge by decreasing minute ventilation, \dot{V}_{E}, of the patient in order to increase PaCO₂ levels. For instance, with reference to the equation above, \dot{V}_{E} may be reduced by decreasing RR and/or by decreasing V_{T}. It may be preferable to reduce \dot{V}_{E} by decreasing the RR. According to embodiments, minute ventilation may be reduced to achieve a target PaCO₂ level. Alternatively, according to embodiments, minute ventilation may be reduced by a test percentage, e.g., 10%.

For example, when the ventilator is equipped to measure PaCO₂ or a surrogate for PaCO₂, the automatic challenge module 226 may reduce \dot{V}_{E} to achieve a target PaCO₂ level. For example, to achieve a target PaCO₂ level that is about 5% greater than a currently measured PaCO₂ level, \dot{V}_{E} may be reduced by about 5%. In order to reduce \dot{V}_{E}, by about 5%, RR may be decreased by about 5% or V_{T} may be decreased by about 5%. Alternatively, a combined reduction of RR and V_{T} that results in a reduction of \dot{V}_{E} of about 5% may be made. As described above, if the ventilator challenge is unsuccessful, the target PaCO₂ level may be increased for subsequent ventilator challenges, within an acceptable range.

Alternatively, when the ventilator is not equipped to measure PaCO₂ or a surrogate for PaCO₂, the automatic challenge module 226 may decrease \dot{V}_{E}, by a test percentage (e.g., between about 10% and 20%, preferably about 10%). According to embodiments, the test percentage may be selected such that the reduction in ventilation will not be harmful to the patient, but will sufficiently increase PaCO₂ in order to stimulate spontaneous breathing efforts. According to some embodiments, the test percentage by which \dot{V}_{E} is reduced may be selected by a clinician and may be based on any suitable protocol, nomogram, or criteria (e.g., disease state, body temperature, or otherwise), or determined via any suitable equation or calculation (e.g., to achieve a particular PaCO₂ value or otherwise). According to alternative embodiments, the test percentage may be predetermined (e.g., by the ventilator manufacturer, by an institution, etc.) and may be based on any suitable protocol, nomogram, or criteria or determined via any suitable equation or calculation. According to some embodiments, the test percentage may be incrementally increased, within an acceptable range, when a previous ventilator challenge was unsuccessful in stimulating spontaneous breathing efforts.

Alternatively, when the ventilator is not equipped to measure PaCO₂ or a surrogate for PaCO₂, the automatic challenge module 226 may decrease \dot{V}_{E} by a fixed amount (e.g., 2 L/min, 3 L/min, 4 L/min, etc.). According to embodiments, the fixed amount may be selected such that the reduction in ventilation will not be harmful to the patient, but will sufficiently increase PaCO₂ in order to stimulate spontaneous breathing efforts. According to some embodiments, the fixed amount by which \dot{V}_{E} is reduced may be selected by a clinician and may be based on any suitable protocol, nomogram, or criteria or determined via any suitable equation or calculation. According to alternative embodiments, the fixed amount may be predetermined (e.g., by the ventilator manufacturer, by an institution, etc.) and may be based on any suitable protocol, nomogram, or criteria or determined via any suitable equation or calculation. According to some embodiments, the fixed amount may be incrementally increased, within an acceptable range, when a previous ventilator challenge was unsuccessful in stimulating spontaneous breathing efforts.

According to other embodiments, in cases where the ventilator is either equipped or not to measure PaCO₂ or a surrogate for PaCO₂, the automatic challenge module 226 may decrease ventilation for a period of time (e.g., between about 2 minutes and 8 minutes, preferably 5 minutes). According to embodiments, the period of time is selected such that it is sufficient to reduce ventilation by a specified amount (e.g., to achieve a target PaCO₂ level or to reduce \dot{V}_{E} by a test percentage), but such that the period of time at the reduced \dot{V}_{E} is not harmful to the patient. According to some embodiments, the period of time may be selected by the clinician and may be based on any suitable protocol or criteria or may be determined via any suitable equation or calculation. According to other embodiments, the period of time may be predetermined (e.g., by the manufacturer or an institution) and may be based on any suitable protocol or criteria or may be determined via any suitable equation or calculation. According to some embodiments, the period of time for a ventilator challenge may be incrementally increased for a subsequent ventilator challenge, within an acceptable range, when a previous ventilator challenge was unsuccessful in stimulating spontaneous breathing efforts. According to other embodiments, a same period of time may be maintained for subsequent ventilator challenges whether or not the previous ventilator challenge was unsuccessful in stimulating spontaneous breathing efforts. According to embodiments, when the period of time for the ventilator challenge expires, minute ventilation is returned to pre-challenge levels (e.g., according to the baseline ventilation settings).

According to other embodiments, in cases where the ventilator is either equipped or not to measure PaCO₂ or a surrogate for PaCO₂, the automatic challenge module 226 may cancel a ventilator challenge before the period of time expires when various other threshold conditions are met, e.g., low oxygen saturation (SpO₂), increased heart rate, or any indication that the patient is responding adversely to the reduction in ventilation.

According to other embodiments, in cases where the ventilator is either equipped or not to measure PaCO₂ or a surrogate for PaCO₂, the automatic challenge module 226 may provide a ventilator challenge on an automatic, periodic basis. That is, according to embodiments, a ventilator challenge may be automatically provided by the ventilator based on a set interval. Automatically, as used in this embodiment, means without clinician input. The set interval may dictate a time between ventilator challenges. For example, the set interval may be every 30 minutes, every 60 minutes, every 90 minutes, every 120 minutes, or any other suitable interval (preferably every 30 minutes). According to embodiments, the set interval may be input by the clinician or may be predetermined (e.g., by the ventilator manufacturer, by an institution, etc.). According to embodiments, the set interval may be adjusted at any time during ventilation by the clinician. According to some embodiments, the set interval may be incrementally decreased. For example, when a previous ventilator challenge was unsuccessful in stimulating spontaneous breathing efforts, ventilator challenges may be provided more often, within an acceptable range. According to other embodiments, the set interval may be incrementally increased. For example, when the patient shows signs of exhaustion or other deleterious effect, ventilator challenges may be provided less often, within an acceptable range. According to other embodiments, a same set interval may be maintained for subsequent ventilator challenges.

According to a non-limiting example, the automatic challenge module 226 may decrease \dot{V}_{E} by 10% by decreasing RR by 10% or by decreasing V_{T} by 10%. For example, if \dot{V}_{E} is 10 L/min (e.g., 0.5 L/breath*20 breaths/min), \dot{V}_{E} may be reduced by 10% (i.e., to 9 L/min) by reducing the RR by 10% (to 18 breaths/min). Alternatively, \dot{V}_{E} may be reduced by 10% (i.e., to 9 L/min) by reducing the V_{T} by 10% (to 0.45 L/breath). A reduction in \dot{V}_{E} by 10% will result in about a 10% increase in PaCO₂ (e.g., from about 40 mm Hg to about 44 mm Hg). Note that this non-limiting example is provided for illustrative purposes only and values chosen for \dot{V}_{E}, V_{T}, and RR may or may not be representative of actual patient settings according to the present disclosure.

Ventilator 202 may further include a patient monitor module 228. According to embodiments, patient monitor module 228 may be responsible for detecting spontaneous patient effort and, if available, monitoring patient PaCO₂ or a surrogate for PaCO₂. For example, patient monitor module 228 may be configured to detect spontaneous breathing efforts via various methods. The term “triggering” refers to the transition from exhalation to inhalation in order to distinguish it from the transition from inhalation to exhalation (referred to as “cycling”). Thus, a triggering patient refers to a spontaneously breathing patient that is able to initiate the transition from exhalation to inhalation. Alternatively, a non-triggering patient does not exhibit sufficient patient effort to initiate the transition from exhalation to inhalation.

According to embodiments, patient monitor module 228 may detect spontaneous breathing efforts via various methods, e.g., a pressure-triggering method, a flow-triggering method, direct or indirect measurement of nerve impulses, or any other suitable method. Sensing devices utilized by the ventilator to detect spontaneous breathing efforts may be either internal or distributed and may include any suitable sensing device, as described above. In addition, the sensitivity of the ventilator to changes in pressure and/or flow may be adjusted such that the ventilator may accurately detect spontaneous breathing efforts, i.e., the lower the pressure or flow change setting the more sensitive the ventilator may be to patient triggering.

According to embodiments, patient monitor module 228 may detect spontaneous breathing efforts via a pressure-triggering method. The pressure-triggering method may involve the ventilator detecting a slight drop in circuit pressure at the end of exhalation. The slight drop in circuit pressure may indicate that the patient's respiratory muscles are creating a slight negative pressure gradient between the patient's lungs and the airway opening in an effort to inspire. Based on a pressure sensitivity setting, the slight drop in circuit pressure may breach a pressure trigger threshold. The ventilator may interpret the breach of the pressure trigger threshold as a spontaneous breathing effort and may consequently initiate inspiration by delivering respiratory gases.

According to embodiments, patient monitor module 228 may detect spontaneous breathing efforts via a flow-triggering method. Specifically, the ventilator may monitor the circuit flow and may detect a slight drop in flow during exhalation. The slight drop in flow may indicate that the patient is attempting to inspire. In this case, the ventilator is detecting a drop in bias flow (or baseline flow) attributable to a slight redirection of gases into the patient's lungs (in response to a slightly negative pressure gradient as discussed above). Bias flow refers to a constant flow existing in the circuit during exhalation that enables the ventilator to detect expiratory flow changes and patient triggering. For example, whereas gases are generally flowing out of the patient's lungs during exhalation, a drop in flow may occur as some gases are redirected and flow into the lungs in response to the slightly negative pressure gradient between the patient's lungs and the body's surface. Based on a flow sensitivity setting, the slight drop in flow below the bias flow may breach a flow trigger threshold (e.g., 2 L/min below bias flow). The ventilator may interpret the breach of the flow trigger threshold as a spontaneous breathing effort and may consequently initiate inspiration by delivering respiratory gases.

According to some embodiments, the patient monitor module 228 may also be responsible for monitoring patient PaCO₂ or a surrogate for PaCO₂. As such, patient monitor module 228 may be in communication with the carbon dioxide monitor 222 and/or data processing module 224 to retrieve ventilatory data associated with PaCO₂ or a surrogate for PaCO₂. According to embodiments, patient monitor module 228 may correlate ventilatory data associated with PaCO₂ or a surrogate for PaCO₂ with the detection of spontaneous breathing efforts. As such, patient monitor module 228 may identify unsuccessful PaCO₂ levels, successful PaCO₂ levels, and/or the patient-specific PaCO₂ threshold level. Moreover, the correlated ventilatory data may be used to configure new ventilatory settings in order to prevent over-ventilation.

According to other embodiments, data processing module 224 may retrieve data regarding detection of spontaneous breathing efforts from the patient monitor module 228 and may correlate ventilatory data associated with PaCO₂ or a surrogate for PaCO₂ with the detection of spontaneous breathing efforts. In this case, data processing module 224 may identify unsuccessful PaCO₂ levels, successful PaCO₂ levels, and/or the patient-specific PaCO₂ threshold level. Moreover, the correlated ventilatory data may be used to configure new ventilatory settings s in order to prevent over-ventilation.

As should be appreciated, the various modules described above do not represent an exclusive array of modules. Indeed, any number of additional modules may be suitably configured to execute one or more of the above-described operations within the spirit of the present disclosure. Furthermore, the various modules described above do not represent a necessary array of modules. Indeed, any number of the disclosed modules may be appropriately replaced by other suitable modules without departing from the spirit of the present disclosure. According to some embodiments, operations executed by the various modules described above may be stored as computer-executable instructions in the ventilator memory, e.g., memory 112, which computer-executable instructions may be executed by one or more processors, e.g., processors 116, of the ventilator.

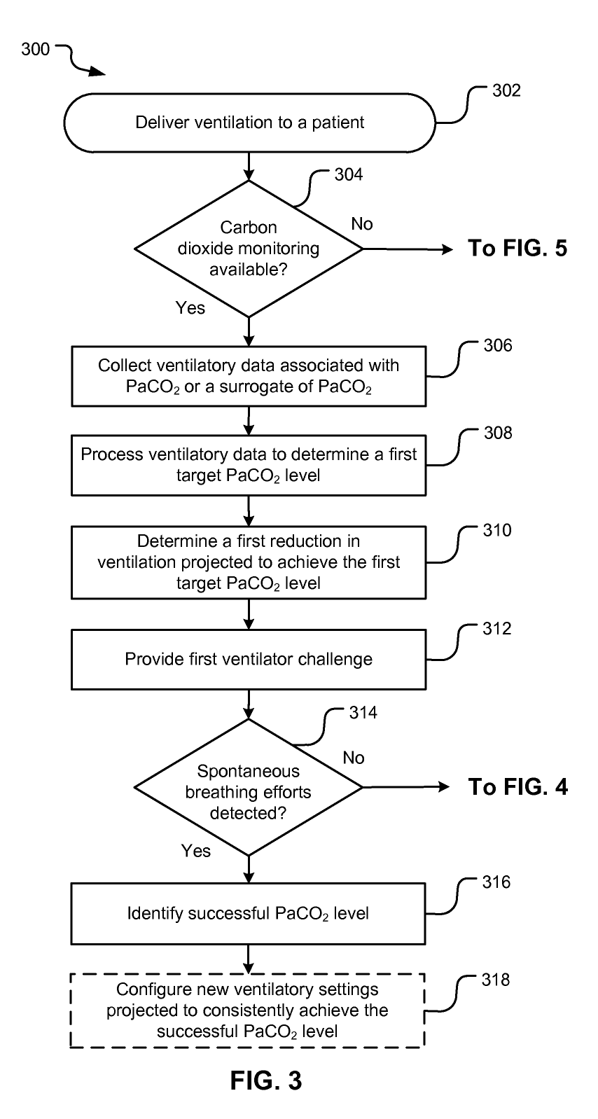

FIG. 3 is a flow chart illustrating an embodiment of a method for providing a first ventilator challenge to stimulate spontaneous breathing efforts when carbon dioxide monitoring is available. That is, the illustrated embodiment of the method 300 depicts a method for providing a first ventilator challenge when ventilatory data associated with PaCO₂ or a surrogate for PaCO₂ is available, as described above.

Method 300 begins with deliver ventilation operation 302. According to embodiments, ventilation may include mandatory ventilation or spontaneous ventilation. Mandatory ventilation refers to set amount of ventilation that is provided on a predetermined schedule, whereas spontaneous ventilation refers to set or variable amount of ventilation provided on a patient-determined schedule. According to embodiments, the present system and methods may be more appropriate in a mandatory ventilation setting in which the patient consistently fails to initiate spontaneous breathing efforts. However, it is envisioned that the present systems and methods may also be useful when a patient who has been receiving spontaneous ventilation fails to initiate spontaneous breathing efforts for some period of time such that mandatory ventilation has resumed. In this case, a ventilator challenge may be provided to stimulate the patient to return to spontaneous breathing.

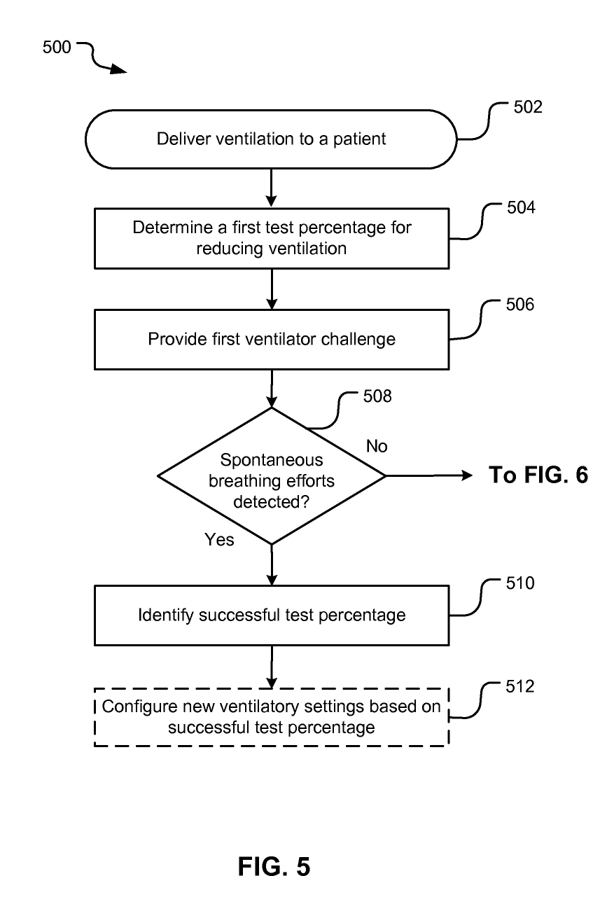

At first determination operation 304, the ventilator may determine whether the ventilator is configured to monitor carbon dioxide. As described above, the ventilator may be configured to monitor carbon dioxide using a number of monitoring devices. For example, the ventilator may be configured to monitor PaCO₂ or a surrogate for PaCO₂. If carbon dioxide monitoring is available, the method proceeds to collect ventilatory data operation 306. Alternatively, if carbon dioxide monitoring is not available, the method proceeds to FIG. 5.

At collect ventilatory data operation 306, the ventilator may conduct various data processing operations. For example, the ventilator may collect or derive various ventilatory data associated with PaCO₂ and/or one or more surrogates for PaCO₂. For example, as described above, the ventilator may directly collect ventilatory data associated with PaCO₂. Additionally or alternatively, the ventilator may indirectly derive ventilatory data associated with PaCO₂ based on collecting ventilatory data associated with surrogates for PaCO₂. Surrogates for PaCO₂ may include, inter alia, EtCO₂, PtcCO₂, PvCO₂, P_{A}CO₂, or any other suitable measurement of carbon dioxide.

At process ventilatory data operation 308, the ventilator may determine a first target PaCO₂ level. According to embodiments, the ventilator may determine the first target PaCO₂ level based on a comparison of the collected and/or derived ventilatory data associated with PaCO₂ and average patient-specific PaCO₂ threshold levels based on any suitable clinical study, protocol, nomogram, or otherwise (e.g., average patient-specific PaCO₂ threshold levels based on patient IBW, patient diagnosis, patient disease state, patient body temperature, patient gender, patient age, etc.). According to other embodiments, the first target PaCO₂ level may be selected by the clinician based on any suitable clinical study, protocol, nomogram, or otherwise.

At determine reduction in ventilation operation 310, the ventilator may determine a first reduction in ventilation that is projected to achieve the first target PaCO₂ level. For example, if the first target PaCO₂ level is greater than current values for PaCO₂ by a certain percentage, ventilation may be reduced by the certain percentage such that the corresponding increase in PaCO₂ is projected to achieve the first target PaCO₂ level. According to some embodiments, ventilation is reduced by decreasing minute ventilation, \dot{V}_{E}. For example, to achieve a first target PaCO₂ level that is about 5% greater than a current PaCO₂ level, \dot{V}_{E}, may be reduced by about 5% (i.e., the first reduction in ventilation). In order to reduce \dot{V}_{E} by about 5%, RR may be decreased by about 5% or V_{T} may be decreased by about 5%. Alternatively, a combined decrease in RR and V_{T} that results in a reduction of \dot{V}_{E} by about 5% may be made.

At provide ventilator challenge operation 312, the ventilator may provide a first ventilator challenge for stimulating spontaneous breathing efforts. According to embodiments, the first ventilator challenge may be provided by decreasing RR and/or V_{T} to result in the first reduction in ventilation. According to embodiments, the first ventilator challenge may be conducted for a first period of time (e.g., between about 2 minutes and 8 minutes, preferably 5 minutes). According to other embodiments, the first ventilator challenge may be cancelled before the first period of time expires when various other threshold conditions are met, e.g., low SpO₂, increased heart rate, or any indication that the patient is responding adversely to the first ventilator challenge. According to embodiments, the first period of time may be selected such that it is sufficient to cause the first reduction in ventilation, but such that the first period of time at the reduced ventilation is not harmful to the patient. According to some embodiments, the first period of time may be selected by the clinician. According to other embodiments, the first period of time may be predetermined (e.g., by the manufacturer or an institution). According to embodiments, when the first period of time for the first ventilator challenge expires, minute ventilation is returned to pre-challenge levels as configured by the baseline ventilatory settings.

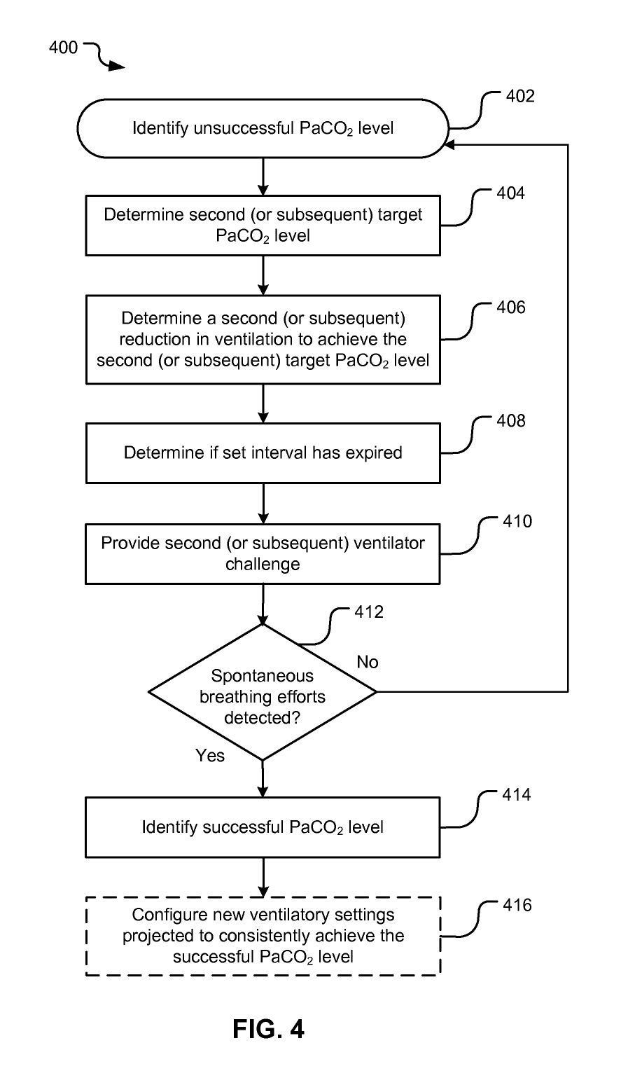

At second determination operation 314, the ventilator may determine whether spontaneous breathing efforts were detected in response to the first ventilator challenge. According to embodiments, the ventilator may detect spontaneous breathing efforts via various methods, e.g., a pressure-triggering method, a flow-triggering method, direct or indirect measurement of nerve impulses, or any other suitable method. If spontaneous breathing efforts were detected, the method may proceed to identify successful PaCO₂ level operation 316. Alternatively, if spontaneous breathing efforts were not detected, the method may proceed to FIG. 4.

At identify successful PaCO₂ level operation 316, the ventilator may store data associated with a successful PaCO₂ level. A successful PaCO₂ level is a PaCO₂ level that was concomitant with a ventilator challenge that induced spontaneous breathing efforts. For example, the ventilator may store data associated with a reduction in ventilation that gave rise to the successful PaCO₂ level. According to embodiments, the reduction in ventilation may be a reduction in \dot{V}_{E} that is associated with a decrease in RR and/or V_{T}. According to embodiments, the ventilator may provide subsequent ventilator challenges that target the successful PaCO₂ level to stimulate spontaneous breathing efforts. According to embodiments, subsequent ventilator challenges that target the successful PaCO₂ level may provide the reduction in ventilation that gave rise to the successful PaCO₂ level.

At optional configure new ventilatory settings operation 318, the ventilator may configure new ventilatory settings that generally reduce ventilation over the baseline ventilation settings such that the non-triggering patient is not over-ventilated and such that the successful PaCO₂ level may be consistently achieved. According to embodiments, when the successful PaCO₂ level is consistently achieved, the patient may consistently initiate spontaneous breathing efforts. According to some embodiments, the new ventilatory settings may be configured by the clinician. According to other embodiments, the new ventilatory settings may be automatically adjusted by the ventilator. According to some embodiments, the ventilator may discontinue providing ventilator challenges when new ventilatory settings are configured unless and until the patient fails to initiate spontaneous breathing efforts.

As should be appreciated, the particular steps and methods described above with reference to FIG. 3 are not exclusive and, as will be understood by those skilled in the art, the particular ordering of steps as described herein is not intended to limit the method, e.g., steps may be performed in differing order, additional steps may be performed, and disclosed steps may be excluded without departing from the spirit of the present methods.

FIG. 4 is a flow chart illustrating an embodiment of a method for providing a second ventilator challenge to stimulate spontaneous breathing efforts when carbon dioxide monitoring is available. Method 400 begins when spontaneous breathing efforts were not detected after a first ventilator challenge.

At identify unsuccessful PaCO₂ level operation 402, the ventilator may store data associated with an unsuccessful PaCO₂ level. An unsuccessful PaCO₂ level is a PaCO₂ level that was concomitant with a ventilator challenge that did not induce spontaneous breathing efforts. For example, the ventilator may store data associated with a reduction in ventilation that gave rise to the unsuccessful PaCO₂ level. According to embodiments, the reduction in ventilation may be a reduction in \dot{V}_{E} that is associated with a decrease in RR and/or V_{T}.

At determine target PaCO₂ level operation 404, the ventilator may determine a second (or subsequent) target PaCO₂ level. According to embodiments, the ventilator may determine the second (or subsequent) target PaCO₂ level based on the unsuccessful PaCO₂ level. That is, the second (or subsequent) target PaCO₂ level may be greater than the unsuccessful PaCO₂ level by an incremental amount. The incremental amount may be any suitable amount such that the second (or subsequent) target PaCO₂ level is within an acceptable range. The acceptable range for target PaCO₂ levels may be based on the patient's disease state or other appropriate criteria.

At determine reduction in ventilation operation 406, the ventilator may determine a second (or subsequent) reduction in ventilation that is projected to achieve the second (or subsequent) target PaCO₂ level. For example, if the second (or subsequent) target PaCO₂ level is greater than current values for PaCO₂ by a certain percentage, ventilation may be reduced by the certain percentage such that the corresponding increase in PaCO₂ is projected to achieve the second (or subsequent) target PaCO₂ level. According to some embodiments, ventilation is reduced by decreasing minute ventilation, \dot{V}_{E}. For example, to achieve a second (or subsequent) target PaCO₂ level that is about 6% greater than a current PaCO₂ level, \dot{V}_{E} may be reduced by about 6%. In order to reduce \dot{V}_{E} by about 6%, RR may be decreased by about 6% or V_{T} may be decreased by about 6%. Alternatively, a combined decrease in RR and V_{T} that results in a reduction of \dot{V}_{E} by about 6% may be made.

At determine set interval operation 408, the ventilator may determine whether a set interval has elapsed since the first ventilator challenge (or a previous ventilator challenge). The set interval may dictate a time between ventilator challenges. For example, the set interval may be every 30 minutes, every 60 minutes, every 90 minutes, every 120 minutes, or any other suitable interval (preferably every 30 minutes). According to embodiments, the set interval may be input by the clinician or may be predetermined. According to some embodiments, the set interval may be adjusted at any time during ventilation by the clinician. According to some embodiments, the set interval may be incrementally increased, incrementally decreased, or maintained as desired. Upon determining that the set interval has elapsed, the methods may proceed to ventilator challenge operation 410. Upon determining that the set interval has not elapsed, the method may return to determine set interval operation 408.

At provide ventilator challenge operation 410, the ventilator may provide a second (or subsequent) ventilator challenge for stimulating spontaneous breathing efforts. According to embodiments, the second ventilator challenge may be provided by reducing \dot{V}_{E} by decreasing RR and/or V_{T} in order to stimulate spontaneous breathing efforts. According to embodiments, the second (or subsequent) ventilator challenge may be conducted for a second period of time (e.g., between about 2 minutes and 8 minutes, preferably 5 minutes). According to other embodiments, the second ventilator challenge may be cancelled before the second period of time expires when various other threshold conditions are met, e.g., low SpO₂, increased heart rate, or any indication that the patient is responding adversely to the second (or subsequent) ventilator challenge. According to embodiments, the second period of time may be selected such that it is sufficient to cause the second reduction in ventilation, but such that the second period of time at the reduced \dot{V}_{E} is not harmful to the patient. According to some embodiments, the second period of time for a second (or subsequent) ventilator challenge may be incrementally increased over the first (or previous) period of time, within an acceptable range, when a previous ventilator challenge was unsuccessful in stimulating spontaneous breathing efforts. According to alternative embodiments, the second period of time may be the same or less than the first period of time.

At determination operation 412, the ventilator may determine whether spontaneous breathing efforts were detected during or after the second (or subsequent) ventilator challenge. According to embodiments, the ventilator may detect spontaneous breathing efforts via various methods, e.g., a pressure-triggering method, a flow-triggering method, direct or indirect measurement of nerve impulses, or any other suitable method. If spontaneous breathing efforts were detected, the method may proceed to identify successful PaCO₂ level operation 414. Alternatively, if spontaneous breathing efforts were not detected, the method may return to identify unsuccessful PaCO₂ level operation 402.

At identify successful PaCO₂ level operation 414, the ventilator may store data associated with a successful PaCO₂ level. A successful PaCO₂ level is a PaCO₂ level that was concomitant with a ventilator challenge that induced spontaneous breathing efforts. For example, the ventilator may store data associated with a reduction in ventilation that gave rise to the successful PaCO₂ level. According to embodiments, the reduction in ventilation may be a reduction in \dot{V}_{E} that is associated with a decrease in RR and/or V_{T}. According to embodiments, the ventilator may provide subsequent ventilator challenges that target the successful PaCO₂ level to stimulate spontaneous breathing efforts. According to embodiments, subsequent ventilator challenges that target the successful PaCO₂ level may provide the reduction in ventilation that gave rise to the successful PaCO₂ level.

At optional configure new ventilatory settings operation 416, the ventilator may configure new ventilatory settings that generally reduce ventilation over the baseline ventilation settings such that the non-triggering patient is not over-ventilated and such that the successful PaCO₂ level may be consistently achieved. According to embodiments, when the successful PaCO₂ level is consistently achieved, the patient may consistently initiate spontaneous breathing efforts. According to some embodiments, the new ventilatory settings may be configured by the clinician. According to other embodiments, the new ventilatory settings may be automatically adjusted by the ventilator. According to some embodiments, the ventilator may discontinue providing ventilator challenges when new ventilatory settings are configured unless and until the patient fails to initiate spontaneous breathing efforts.

As should be appreciated, the particular steps and methods described above with reference to FIG. 4 are not exclusive and, as will be understood by those skilled in the art, the particular ordering of steps as described herein is not intended to limit the method, e.g., steps may be performed in differing order, additional steps may be performed, and disclosed steps may be excluded without departing from the spirit of the present methods.

FIG. 5 is a flow chart illustrating an embodiment of a method for providing a first ventilator challenge to stimulate spontaneous breathing efforts when carbon dioxide monitoring is not available.

The illustrated embodiment of the method 500 depicts a method for providing a first ventilator challenge to stimulate spontaneous breathing efforts in a patient when ventilatory data associated with PaCO₂ or a surrogate for PaCO₂ is not available, as described above.

Method 500 begins with a deliver ventilation operation 502, as described with reference to deliver ventilation operation 302.

At determine test percentage operation 504, the ventilator may determine a first test percentage for reducing ventilation. According to embodiments, the first test percentage may be selected such that the reduction in ventilation will not be harmful to the patient, but will sufficiently increase PaCO₂ in order to stimulate spontaneous breathing efforts. The first test percentage may be selected by a clinician or may be predetermined by the ventilator. According to some embodiments, the reduction in ventilation may be a reduction in \dot{V}_{E} by the first test percentage. According to embodiments, reducing \dot{V}_{E} by the first test percentage may comprise decreasing RR and/or V_{T} by the first test percentage.

At provide ventilator challenge operation 506, the ventilator may provide a first ventilator challenge for stimulating spontaneous breathing efforts. According to embodiments, the first ventilator challenge may be provided by reducing \dot{V}_{E} by the first test percentage. For example, reducing \dot{V}_{E} by the first test percentage may comprise decreasing RR and/or V_{T} by the first test percentage. According to embodiments, the first ventilator challenge may be conducted for a first period of tune, as described above with reference to provide ventilator challenge operation 312. According to embodiments, when the first period of time for the first ventilator challenge expires, minute ventilation is returned to pre-challenge levels according to the baseline ventilatory settings. According to alternative embodiments, the first ventilator challenge may be cancelled before the first period of time expires when various other threshold conditions are met, e.g., low SpO₂, increased heart rate, or any indication that the patient is responding adversely to the first ventilator challenge.

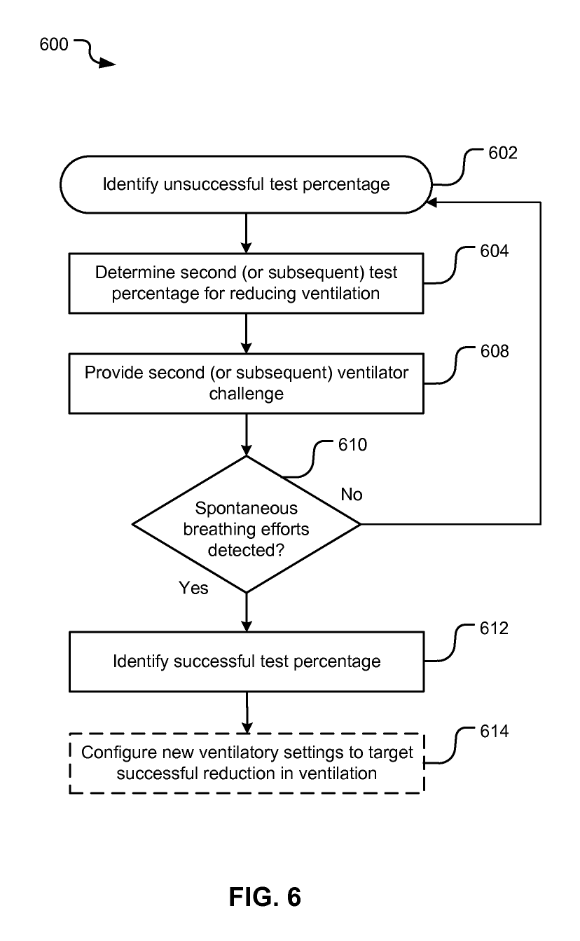

At determination operation 508, the ventilator may determine whether spontaneous breathing efforts were detected in response to the first ventilator challenge. If spontaneous breathing efforts were detected, the method may proceed to identify successful PaCO₂ level operation 510. Alternatively, if spontaneous breathing efforts were not detected, the method may proceed to FIG. 6.

At identify successful test percentage operation 510, the ventilator may store a successful test percentage. A successful test percentage is a test percentage that was used to reduce \dot{V}_{E} in a ventilator challenge that induced spontaneous breathing efforts. According to embodiments, the ventilator may provide subsequent ventilator challenges using the successful test percentage to stimulate spontaneous breathing efforts.

At optional configure new ventilatory settings operation 512, the ventilator may configure new ventilatory settings that generally reduce ventilation over baseline ventilatory settings by the successful test percentage. According to some embodiments, the new ventilatory settings may be adjusted by the clinician. According to other embodiments, the new ventilatory settings may be automatically adjusted by the ventilator. According to some embodiments, the ventilator may discontinue providing ventilator challenges when new ventilatory settings are configured unless and until the patient fails to initiate spontaneous breathing efforts.

As should be appreciated, the particular steps and methods described above with reference to FIG. 5 are not exclusive and, as will be understood by those skilled in the art, the particular ordering of steps as described herein is not intended to limit the method, e.g., steps may be performed in differing order, additional steps may be performed, and disclosed steps may be excluded without departing from the spirit of the present methods.

FIG. 6 is a flow chart illustrating an embodiment of a method for providing a second ventilator challenge to stimulate spontaneous breathing efforts when carbon dioxide monitoring is not available. Method 600 begins when spontaneous breathing efforts were not detected after a first ventilator challenge.

At identify unsuccessful test percentage operation 602, the ventilator may store an unsuccessful test percentage. An unsuccessful test percentage is a test percentage that was used to reduce \dot{V}_{E}, in a ventilator challenge that failed to induce spontaneous breathing efforts.

At determine test percentage operation 604, the ventilator may determine a second (or subsequent) test percentage for reducing ventilation. According to some embodiments, the second (or subsequent) test percentage may be greater than the unsuccessful test percentage by an incremental amount. The incremental amount may be any suitable amount such that the second (or subsequent) test percentage is within an acceptable range. The acceptable range for a test percentage may be based on a projected reduction in \dot{V}_{E} based on the patient's disease state or other appropriate criteria.

At provide ventilator challenge operation 608, the ventilator may provide a second (or subsequent) ventilator challenge for stimulating spontaneous breathing efforts. According to embodiments, the second (or subsequent) ventilator challenge may be provided by reducing \dot{V}_{E} by the second test percentage. For example, reducing \dot{V}_{E} by the second test percentage may comprise decreasing RR and/or V_{T} by the second test percentage. According to embodiments, the second ventilator challenge may be conducted for a second period of time, as described above with reference to provide ventilator challenge operation 312. According to some embodiments the second period of time for a second (or subsequent) ventilator challenge after an unsuccessful challenge may be increased by an incremental amount. According to other embodiments, the second period of time may be the same or less than the first period of time. According to embodiments, when the second period of time for the second (or subsequent) ventilator challenge expires, minute ventilation is returned to pre-challenge levels according to baseline ventilatory settings. According to other embodiments, the second ventilator challenge may be cancelled before the second period of time expires when various other threshold conditions are met, e.g., low SpO₂, increased heart rate, or any indication that the patient is responding adversely to the second (or subsequent) ventilator challenge.

At determination operation 610, the ventilator may determine whether spontaneous breathing efforts were detected in response to the second (or subsequent) ventilator challenge. If spontaneous breathing efforts were detected, the method may proceed to identify successful test percentage operation 612. Alternatively, if spontaneous breathing efforts were not detected, the method may return to identify unsuccessful test percentage operation 602.

At identify successful test percentage operation 612, the ventilator may store a successful test percentage. A successful test percentage is a test percentage that was used to reduce \dot{V}_{E} in a ventilator challenge that induced spontaneous breathing efforts. According to embodiments, the ventilator may provide subsequent ventilator challenges using the successful test percentage to stimulate spontaneous breathing efforts.

At optional configure new ventilatory settings operation 614, the ventilator may configure new ventilatory settings that generally reduce ventilation over baseline ventilatory settings by the successful test percentage to prevent over-ventilation and to promote spontaneous breathing efforts. According to some embodiments, the new ventilatory settings may be adjusted by the clinician. According to other embodiments, the new ventilatory settings may be automatically adjusted by the ventilator. According to some embodiments, the ventilator may discontinue providing ventilator challenges when new ventilatory settings are configured unless and until the patient fails to initiate spontaneous breathing efforts.

As should be appreciated, the particular steps and methods described above with reference to FIG. 6 are not exclusive and, as will be understood by those skilled in the art, the particular ordering of steps as described herein is not intended to limit the method, e.g., steps may be performed in differing order, additional steps may be performed, and disclosed steps may be excluded without departing from the spirit of the present methods.

Unless otherwise indicated, all numbers expressing measurements, dimensions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure. Further, unless otherwise stated, the term “about” shall expressly include “exactly,” consistent with the discussions regarding ranges and numerical data. Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 4 percent to about 7 percent” should be interpreted to include not only the explicitly recited values of about 4 percent to about 7 percent, but also include individual values and sub-ranges within the indicated range. Thus, included in this numerical range are individual values such as 4.5, 5.25 and 6 and sub-ranges such as from 4-5, from 5-7, and from 5.5-6.5, etc. This same principle applies to ranges reciting only one numerical value. Furthermore, such an interpretation should apply regardless of the breadth of the range or the characteristics being described.

It will be clear that the systems and methods described herein are well adapted to attain the ends and advantages mentioned as well as those inherent therein. Those skilled in the art will recognize that the methods and systems within this specification may be implemented in many manners and as such is not to be limited by the foregoing exemplified embodiments and examples. In other words, functional elements being performed by a single or multiple components, in various combinations of hardware and software, and individual functions can be distributed among software applications at either the client or server level. In this regard, any number of the features of the different embodiments described herein may be combined into one single embodiment and alternative embodiments having fewer than or more than all of the features herein described are possible.

While various embodiments have been described for purposes of this disclosure, various changes and modifications may be made which are well within the scope of the present disclosure. Numerous other changes may be made which will readily suggest themselves to those skilled in the art and which are encompassed in the spirit of the disclosure and as defined in the appended claims.

What is claimed is:

1. A ventilator-implemented method for stimulating spontaneous breathing efforts when carbon dioxide monitoring is available, the method comprising:
collecting ventilatory data associated with PaCO₂ or a surrogate for PaCO₂;
processing the ventilatory data to determine a first target PaCO₂ level;
determining a first reduction in ventilation projected to achieve the first target PaCO₂ level;
providing a first ventilator challenge based on the first reduction in ventilation;
determining whether spontaneous breathing efforts are detected in response to the first ventilator challenge; and
when spontaneous breathing efforts are detected, identifying a successful PaCO₂ level associated with the first ventilator challenge.

2. The method of claim 1, wherein the first ventilator challenge is provided until a first period of time expires or a threshold condition is detected.

3. The method of claim 1, wherein the first reduction in ventilation comprises a first reduction in minute ventilation, \dot{V}_{E}, and wherein the first reduction in \dot{V}_{E} comprises a reduction of at least one of: respiratory rate (RR) and tidal volume (V_{T}).

4. The method of claim 1, wherein when spontaneous breathing efforts are not detected, the method further comprises:
identifying the first target PaCO₂ level as an unsuccessful PaCO₂ level; and
determining a second target PaCO₂ level higher than the unsuccessful PaCO₂ level.

5. The method of claim 4, further comprising determining a second reduction in ventilation projected to achieve the second target PaCO₂ level.

6. The method of claim 5, further comprising determining whether a set interval after the first ventilator challenge has expired.

7. The method of claim 6, further comprising:
when the set interval after the first ventilator challenge has expired, providing a second ventilator challenge based on the second reduction in ventilation.

8. The method of claim 7, further comprising:
determining whether spontaneous breathing efforts are detected in response to the second ventilator challenge; and
when spontaneous breathing efforts are detected, identifying a successful PaCO₂ level associated with the second ventilator challenge.

9. A ventilator-implemented method for stimulating spontaneous breathing efforts when carbon dioxide monitoring is not available, the method comprising:
determining a first test percentage for reducing ventilation;
providing a first ventilator challenge based on the first test percentage for reducing ventilation;
determining whether spontaneous breathing efforts are detected in response to the first ventilator challenge; and
when spontaneous breathing efforts are detected, identifying a successful test percentage associated with the first ventilator challenge.

10. The method of claim 9, wherein providing the first ventilator challenge comprises reducing minute ventilation, \dot{V}_{E}, by the first test percentage, wherein reducing \dot{V}_{E} by the first test percentage comprises reducing one of respiratory rate (RR) and tidal volume (V_{T}) by the first test percentage.

11. The method of claim 9, wherein when spontaneous breathing efforts are not detected, the method further comprises:
identifying the first test percentage as an unsuccessful test percentage; and
determining a second test percentage greater than the unsuccessful test percentage for reducing ventilation in a second ventilator challenge.

12. The method of claim 11, further comprising:
determining that a set interval after the first ventilator challenge has expired; and
providing a second ventilator challenge by reducing ventilation by the second test percentage.

13. The method of claim 12, further comprising:
determining whether spontaneous breathing efforts are detected in response to the second ventilator challenge; and
when spontaneous breathing efforts are detected, identifying a successful test percentage associated with the second ventilator challenge.

14. The method of claim 9, wherein the first ventilator challenge is provided for a first period of time.

15. The method of claim 12, wherein the second ventilator challenge is provided for a second period of time.

16. The method of claim 15, wherein the first period of time and the second period of time are different.

17. A ventilator processing interface for stimulating spontaneous breathing efforts, comprising:
means for determining a first test percentage for reducing ventilation to stimulate spontaneous breathing efforts;
means for providing a first ventilator challenge based on the first test percentage for reducing ventilation;
means for determining whether spontaneous breathing efforts are detected in response to the first ventilator challenge; and
when spontaneous breathing efforts are detected, means for identifying a successful test percentage associated with the first ventilator challenge.

18. The ventilator processing interface of claim 17, wherein the first ventilator challenge is provided for a first period of time.

19. The ventilator processing interface of claim 17, wherein the means for providing the first ventilator challenge comprises means for reducing minute ventilation, \dot{V}_{E}, by the first test percentage, wherein the means for reducing \dot{V}_{E} by the first test percentage comprises means for reducing one of respiratory rate (RR) and tidal volume (V_{T}) by the first test percentage.

20. The ventilator processing interface of claim 19, wherein when spontaneous breathing efforts are not detected, the method further comprises:
means for identifying the first test percentage as an unsuccessful test percentage;
means for determining a second test percentage greater than the unsuccessful test percentage for reducing ventilation in a second ventilator challenge; and
means for providing a second ventilator challenge based on the second test percentage for reducing ventilation.
